(19) United States
(12) Patent Application Publication
(10) Pub. No.: US 2023/0008492 A1
(43) Pub. Date: Jan. 12, 2023

(54) AGGREGATION OF UNCONSCIOUS AND CONSCIOUS BEHAVIORS FOR RECOMMENDATIONS AND AUTHENTICATION

(71) Applicant: AT&T Intellectual Property I, L.P., Atlanta, GA (US)

(72) Inventors: Jean-Francois Paiement, Palm Desert, CA (US); Zhengyi Zhou, Chappaqua, NY (US); Eric Zavesky, Austin, TX (US)

(73) Assignee: AT&T Intellectual Property I, L.P., Atlanta, GA (US)

(21) Appl. No.: 17/369,034

(22) Filed: Jul. 7, 2021

(51) Int. Cl. A61B 5/16; A61B 5/00

(52) U.S. Cl. CPC A61B 5/165; A61B 5/6801; A61B 5/6846; A61B 2503/12

(57) ABSTRACT

A system may use unconscious behaviors and conscious behaviors for recommendations and authentication. A method, system, computer readable storage medium, or apparatus provides for sending stimulus, wherein the stimulus is in the presence of an object, wherein the stimulus comprises video, audio, or text; observing activity of the object, wherein the object comprises human or animal; measuring reaction of the object to the stimulus; classifying the reaction of the object to the stimulus; and transmitting a message based on the classification.

### BACKGROUND

A micro expression is a facial expression that only lasts for a short moment. It may be considered the innate result of a voluntary and an involuntary emotional response occurring simultaneously and conflicting with one another, and may occur when the amygdala (the emotion center of the brain) responds appropriately to the stimuli that the individual experiences. In some instances, the individual wishes to conceal this specific emotion. This may result in the individual very briefly displaying true emotions followed by a false emotional reaction.

Human emotions may be considered an unconscious biopsychosocial reaction that derives from the amygdala and typically last 0.5-4.0 seconds, although a micro expression will typically last less than .5 seconds. Unlike regular facial expressions it is very difficult to hide micro expression (e.g., unconscious) reactions. Micro expressions have a low probability of being controlled as they happen in a fraction of a second, but it is possible to capture someone's expressions with a high-speed camera and replay them at much slower speeds. Micro expressions commonly show the following emotions: disgust, anger, fear, sadness, happiness, contempt, and surprise, as well as a wide range of positive and negative emotions not all of which are encoded in facial muscles.

This background information is provided to reveal information believed by the applicant to be of possible relevance. No admission is necessarily intended, nor should be construed, that any of the preceding information constitutes prior art.

### SUMMARY

A system may use unconscious behaviors and conscious behaviors for recommendations and authentication. In an example, an apparatus may include a processor and a memory coupled with the processor that effectuates operations. The operations may include sending stimulus, wherein the stimulus is in the presence of a person, wherein the stimulus comprises video, audio, or text; observing activity of the person; measuring reaction of the object to the stimulus; classifying the reaction of the object to the stimulus; and transmitting a message based on the classification.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE DRAWINGS

Reference will now be made to the accompanying drawings, which are not necessarily drawn to scale.

FIG. 1 illustrates an exemplary system that may use unconscious behaviors and conscious behaviors for recommendations, authentication, or other actions.

FIG. 2 illustrates an exemplary method that may use unconscious behaviors and conscious behaviors for recommendations, authentication, or other actions.

FIG. 3 illustrates an exemplary method that may use unconscious behaviors and conscious behaviors for recommendations, authentication, or other actions.

FIG. 4 illustrates exemplary information used for unconscious behaviors and conscious behaviors for recommendations, authentication, or other actions.

FIG. 5 illustrates a schematic of an exemplary network device.

FIG. 6 illustrates an exemplary communication system that provides wireless telecommunication services over wireless communication networks.

### DETAILED DESCRIPTION

The disclosed subject matter may use micro-expressions and unconscious preferences for authentications, recommendations, or other actions.

Conventional systems may look at standard features for disambiguation for authentication (e.g. background features) and such systems may only consider unalterable user interactions. These systems may focus on singular tasks and not the mix of both conscious and unconscious aspects of a task. Capturing additional information, such as micro expressions, may help clarify or authenticate preferences or identity. The analysis of micro expressions may help capture both conscious and unconscious inclinations.

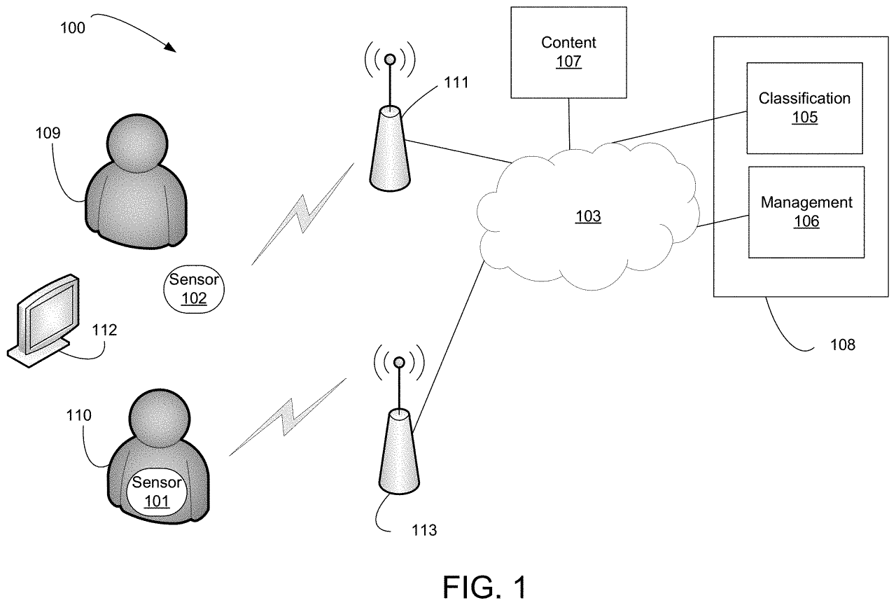

FIG. 1 illustrates an exemplary system that may use unconscious behaviors and conscious behaviors for recommendations, authentication, or other actions. System 100 may include network 103. Sensor 101, sensor 102, base station 111, base station 113, content server 107, display 112, server 108, classification function 105, or management function 106 may be communicatively connected with each other via network 103. Network 103 may include vRouters, access points, DNS servers, firewalls, or the like virtual or physical entities. It is contemplated that the functions disclosed herein may be distributed over multiple physical or virtual entities or located within a single physical or virtual entity. In an example, classification function 105 and management function 106 may be functions located within server 108. Sensor 101 or Sensor 102 may be able to communicate to network 103 through a wired or wireless connection.

Sensor information may be captured by sensor 101, sensor 102, or devices in proximity to a person (e.g., user 109 or user 110). The sensor information may include bio related information (e.g., bio imprints), such as heart beat waves pattern, salinity, pulse, chemical composition of body (e.g., composition of adjacent fluid or tissue), person's voice pattern, person's gait, orientation of sensor 101 (e.g., accelerometer or gyroscope information), audio captured, video captured, or sensed temperature, among other things. The information may include location information (e.g., location imprints). The location information may be determined by the consideration of one or more of the following: global positioning system information, wireless signal strength near sensor 101, wireless signal presence near sensor 101 (e.g., proximate to another sensor, such as sensor 102 which may be implanted or connected with the same or different person), accelerometer information, or gyroscope information, among other things. The information may be recorded over time (e.g., by sensor 101).

In an example scenario, sensor 101 may record video within a time period and send the video to server 108. Server 108 may process the video in a manner to analyze unconscious behaviors (e.g., micro expressions) of one or more objects (e.g., persons or pets) within the video. In this example, the micro expressions may be facial expressions. Micro expressions are usually considered facial expressions that occur within a fraction of a second. This involuntary emotional leakage may expose a person's true emotions. It is contemplated that the movement of other parts of the body may be considered with regard to micro expressions. The stimulus may be provided by content server 107. In this example, the content may be an originally released movie or other content that may have additional stimulus added (e.g., text, video, or audio) by a service provider that was not in the original movie. More generally, stimulus may include audio (e.g., audio alert from mobile device, audio alert from TV, audio alert from smart speaker, audio alert from doorbell system, etc.), video (e.g., text/video alert from mobile device, text/video alert from TV, etc.), or other stimuli (e.g., changes of color or brightness of a display, changed brightness of room lighting, etc.).

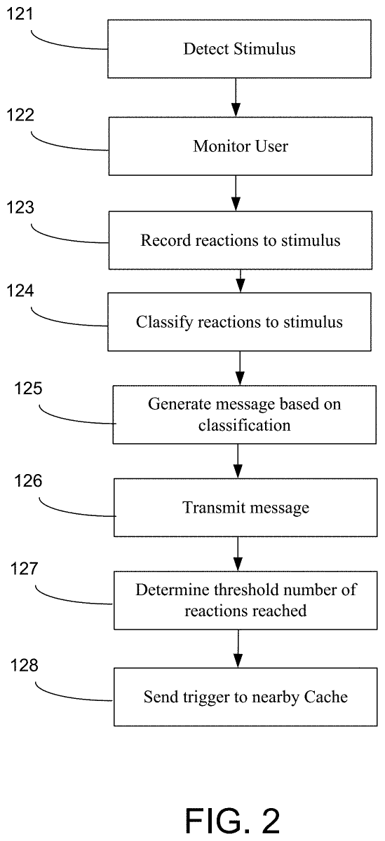

FIG. 2 illustrates an exemplary method that may use unconscious behaviors and conscious behaviors for recommendations, authentication, or other actions.

At step 121, stimulus may be detected. For example, sensor 102 may detect that a movie is being played on a display near user 110 (e.g., a person).

At step 122, the activity of user 110 may be monitored by sensor 101 during the period of the stimulus.

At step 123, reactions of user 110 to the stimulus (e.g., reaction information) may be recorded or otherwise measured.

At step 124, the recorded reactions of step 123 may be classified. For example, classification function 105 may obtain the recorded reactions. Then the audio or video (or other sensor information) of the recorded reactions may be classified based on the analysis of the recording. For example, using machine learning, a first combination of stimuli and a first combination of reactions (e.g., pupil movement in a first direction, dilation, chin movement in a second direction, and an eyebrow movement in a third direction) may be linked to a first micro expression; and a second combination of stimuli and a second combination of reactions may be linked to a second micro expression. Stimulus may be associated with metadata for expected reaction, a general description of the stimulus, or timeline/markers for expected change (i.e., delta) in reaction. In another example, the combination of multiple stimuli of step 121 may be mapping or encoding each of these reactions into a secondary numerical representation that has been learned for each users. For example, machine learning techniques referred to as embedding or encoding may accept the multitude of reactions of step 123 (e.g. pulse, pupil movement, reaction time, etc.) into a numerical array via a learned model. This embedded numerical array allows generalization of the reactions of step 123 of one user to be better classified for the same stimuli of step 121.

At step 125, a message may be generated based on the classification. For example, management function 106 may obtain the classification and connect the classification to an action (e.g., classification trigger action). Such action may include generating or sending a message. The message may be instructions to perform another action which may include sending an alert to a device (e.g., sensor 101, display 112, content server 107, etc.), or changing the type or intensity of stimulus (e.g., content, etc.) near a person, which may include sending instructions to content server 107. Relatively short- or long-term reactions to the stimulus may create a prediction for a specific classification, which may be per user or user cohort. The message may include an indication of authentication

At step 126, the message may be transmitted based on the classification.

At step 127, determining that a threshold number of reactions have occurred and have been classified (e.g., a baseline). Previous personalization models (or profiles) may be included in classification or analysis. Explicit (e.g., conscious) user interaction with system may be utilized as input. In other examples, the threshold may be determined by one or more machine learning methods that have been learned in aggregate across many users or personalized for this specific user. Specifically, models in step 127 may be focused on temporal smoothing of multiple high-frequency messages that may be generated in the system. A large of micro expressions and reactions recorded by sensors 101 is expected per second, so this model may employ one or more smoothing, caching, or aggregation steps during it process of threshold determination. These models add robustness to threshold determination by combining the classification messages of step 125, the user personalization models, and the device or activity of the user (e.g. the context).

At step 128, based on reaching the threshold number, sending classification triggers and corresponding actions to a device near (e.g., in the same room or building) user 110. For example, once a baseline has been established for a recognized person, server 108 may send the classification trigger actions to a local cache near user 110 (e.g., cache of sensor 101, display 112, or base station 113), which may be used to determine the actions for classified micro expressions. Using a local cache may allow for quicker reaction time when unconscious behavior occurs.

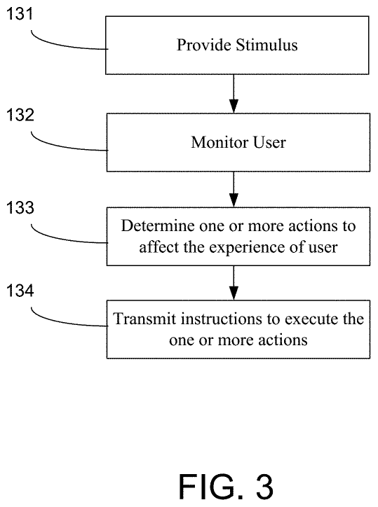

FIG. 3 illustrates an exemplary method that may use unconscious behaviors and conscious behaviors for recommendations, authentication, or other actions.

At step 131, content server 107 may provide stimulus such as video. For this example, user 110 may be viewing a television (TV) show on display 112.

At step 132, user 110 may be monitored. Based on information from sensor 102, for example, it may be determined that user 110 is viewing display 112 in a particular room. In response to the monitoring, sensor information may be obtained regarding user 101. The number and type of reactions of user 110 may be obtained through monitoring. There may be an attempt to proactively determine the model to use for determining actions to affect the experience of user 110 (see step 133). Server 108 may provide a montage of different video or audio stimulus to determine the appropriate model. This montage is not necessarily to just gauge that user 110 likes a particular video clip (or other stimuli), but to gauge characteristics of the video clip that would induce unconscious behavior and use these characteristics to provide the appropriate recommendation, authentication, or other action.

At step 133, determining one or more actions to affect the experience of user 110 based on the monitoring of step 132. The actions to affect experience may be based on a first model (e.g., baseline) associated with users in similar situation (e.g., similar age, room, time period, type of display, etc.) or second model which may be associated with multiple iterations of analyzing unconscious behaviors and sensor information associated with user 110. In a first example, server 108 may determine that user 110 is particularly sensitive (e.g., upsetting) to content with a certain rate of light flashes. In this first example, it may be determined that a warning may need to be sent. There may also be a determination of how the warning is sent (e.g., text on display 112, text and vibration of a wearable, audio, haptic feedback, etc.). Another action for this first example, may be to manipulate how the content is displayed so such flashing is reduced to a level appropriate or user 110 or eliminated.

In a second example, server 108 may determine that user 110 prefers TV shows that are more fast paced or have certain types of background music. Therefore user 110 may be presented different background music (e.g., fast bass rhythms) in a presented TV show than another user (e.g., user 109) would be presented.

The determining may notify a subsequent part of the experience (e.g. automated messaging or alerts), it may alter the current experience (e.g. different steps in the workflow or video playlist), it may alter the intensity of the current experience (e.g. lower volume, suppress upsetting images, etc.) or it may combine the approaches and dynamically evaluate user preferences more interactively (e.g. propose one or more options and allow the user to choose between those options explicitly).

At step 134, transmitting instructions to execute the one or more actions of step 133.

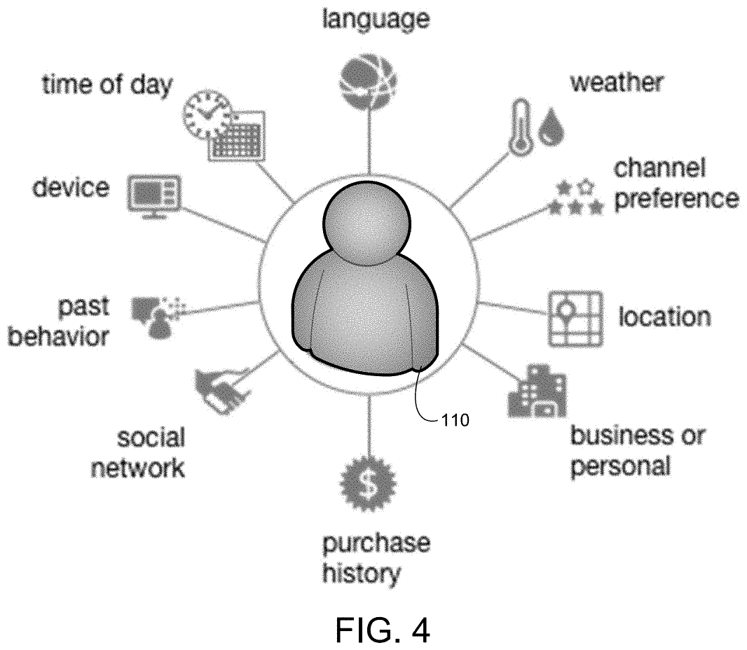

The disclosed systems, methods, and apparatuses may use information regarding unconscious behaviors or conscious behaviors for recommendations, authentication, or other actions. As provided herein, the information obtained may be a mix of high frequency, precognitive signals (e.g. brain or micro-expression reaction), subsequent user actions (spoken sentiment, large facial expression, logging out of app, specific gestures), and other information, such as time of day, weather, or location, among other things (see FIG. 4). The obtained information may be used for authentication services (e.g., for static identity and activities) that uses conscious or unconscious reactions. Discovery of unconscious preferences may be a significant part of identification of a user and may also be utilized with recommendation services. The obtained information may be used for services that analyze or predict a demographic or personal attributes given stimulus (e.g., video content) and the user's reaction (e.g., via neurological -based behavior). Some preferences by a user are unconscious and difficult for them to discover, so the disclosed system may allow for discovery of this unconscious information and use in seemingly unusual situations, such as dating profiles (e.g., there may be generated an unconscious preference score for each profile) or job applications for various professions.

The disclosed system may automatically differentiate recommendations (which may include advertising or alerts) by different context (e.g., work, with friends, at home), behavioral observation (conscious or unconscious), or magnitude of behavioral observation (e.g., passionate, ambivalent, etc.) that may be specifically trained for each user. In an example, the disclosed system may determine who is viewing and using a device. The system may allow for disambiguation between who is watching and who “has the remote” as “primary viewer” by unconscious behavioral differences. In addition, the system may include bias detection (e.g., the user's demeanor generally alone or with other users). The obtained sensor information may be processed in a different way (e.g., aggregated on a different level) and may cause for a modified analysis (e.g., although it appears user 110 enjoyed a movie with multiple people, bias places user 110 as generally optimistic, so the system normalizes the behavioral classification to only mild satisfaction for user 110).

The disclosed system may automatically authenticate a user identity based on behavior during tasks (e.g., continuous tasks) to validate long-term identity for a service. In an example situation regarding account sharing, based on unconscious information (e.g., high pulse) and conscious information (e.g., password), there may be a determination that a user with a high pulse is just excited versus up to something nefarious (e.g. account fraud).

Deeper understanding of user preferences where systems or the user may be unable to adequately express their need, interest, or its magnitude provides a foundation for a rich recommendation system without explicit expression by user (or complementing it). With passive preference determination, a provider can send real-time feedback to content creators and advertisers to improve or otherwise adjust products for a specific user and identity/demographic. Also, the system allows for detection of “tune away” or loss of interest as well as what was a likely distractor for interest (dislike, distraction, etc.). The system allows for combination of multiple methods for authentication (beyond two-factor authentication) that have a rich, hard to emulate process. The system may attempt to evoke specific behavior or reaction from user to provide that expectation as feature input to system (e.g., in the case of fraud detection or normalization estimate).

Additional use cases for the disclosed systems include therapy situations. For example, usage in education and therapy situations (e.g. Cognitive Behavioral Therapy) where the system couples short- and long-term responses with the user's desire to change a response (e.g. afraid of clowns, but gradual exposure strategically embedded in video (e.g., movie or TV) or other stimulus to clown-like items may lead to a reduction in perceived and actual fear).

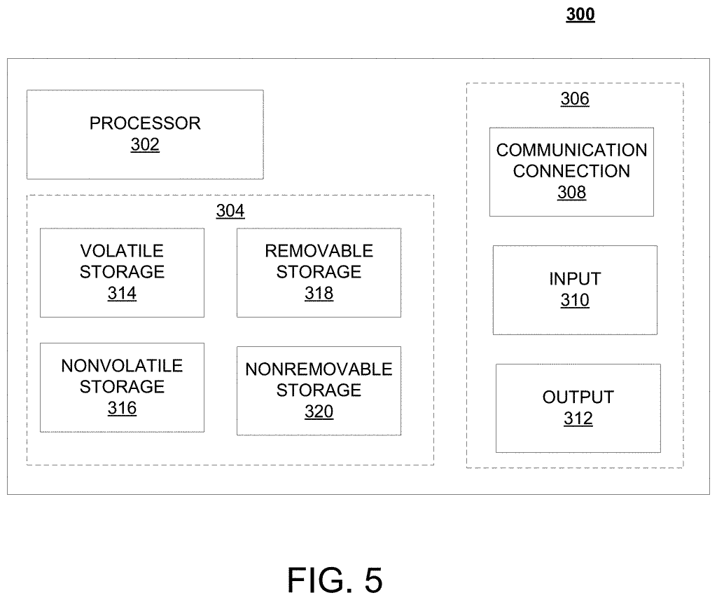

FIG. 5 is a block diagram of network device 300 that may be connected to or comprise a component of system 100. Network device 300 may comprise hardware or a combination of hardware and software. The functionality to facilitate telecommunications via a telecommunications network may reside in one or combination of network devices 300. Network device 300 depicted in FIG. 5 may represent or perform functionality of an appropriate network device 300, or combination of network devices 300, such as, for example, a component or various components of a cellular broadcast system wireless network, a processor, a server, a gateway, a node, a mobile switching center (MSC), a short message service center (SMSC), an automatic location function server (ALFS), a gateway mobile location center (GMLC), a radio access network (RAN), a serving mobile location center (SMLC), or the like, or any appropriate combination thereof. It is emphasized that the block diagram depicted in FIG. 5 is exemplary and not intended to imply a limitation to a specific implementation or configuration. Thus, network device 300 may be implemented in a single device or multiple devices (e.g., single server or multiple servers, single gateway or multiple gateways, single controller or multiple controllers). Multiple network entities may be distributed or centrally located. Multiple network entities may communicate wirelessly, via hard wire, or any appropriate combination thereof.

Network device 300 may comprise a processor 302 and a memory 304 coupled to processor 302. Memory 304 may contain executable instructions that, when executed by processor 302, cause processor 302 to effectuate operations associated with mapping wireless signal strength.

In addition to processor 302 and memory 304, network device 300 may include an input/output system 306. Processor 302, memory 304, and input/output system 306 may be coupled together (coupling not shown in FIG. 5) to allow communications between them. Each portion of network device 300 may comprise circuitry for performing functions associated with each respective portion. Thus, each portion may comprise hardware, or a combination of hardware and software. Input/output system 306 may be capable of receiving or providing information from or to a communications device or other network entities configured for telecommunications. For example, input/output system 306 may include a wireless communications (e.g., 3G/4G/GPS) card. Input/output system 306 may be capable of receiving or sending video information, audio information, control information, image information, data, or any combination thereof. Input/output system 306 may be capable of transferring information with network device 300. In various configurations, input/output system 306 may receive or provide information via any appropriate means, such as, for example, optical means (e.g., infrared), electromagnetic means (e.g., RF, Wi-Fi, Bluetooth®, ZigBee®), acoustic means (e.g., speaker, microphone, ultrasonic receiver, ultrasonic transmitter), or a combination thereof. In an example configuration, input/output system 306 may comprise a Wi-Fi finder, a two-way GPS chipset or equivalent, or the like, or a combination thereof.

Input/output system 306 of network device 300 also may contain a communication connection 308 that allows network device 300 to communicate with other devices, network entities, or the like. Communication connection 308 may comprise communication media. Communication media typically embody computer-readable instructions, data structures, program modules or other data in a modulated data signal such as a carrier wave or other transport mechanism and includes any information delivery media. By way of example, and not limitation, communication media may include wired media such as a wired network or direct-wired connection, or wireless media such as acoustic, RF, infrared, or other wireless media. The term computer-readable media as used herein includes both storage media and communication media. Input/output system 306 also may include an input device 310 such as keyboard, mouse, pen, voice input device, or touch input device. Input/output system 306 may also include an output device 312, such as a display, speakers, or a printer.

Processor 302 may be capable of performing functions associated with telecommunications, such as functions for processing broadcast messages, as described herein. For example, processor 302 may be capable of, in conjunction with any other portion of network device 300, determining a type of broadcast message and acting according to the broadcast message type or content, as described herein.

Memory 304 of network device 300 may comprise a storage medium having a concrete, tangible, physical structure. As is known, a signal does not have a concrete, tangible, physical structure. Memory 304, as well as any computer-readable storage medium described herein, is not to be construed as a signal. Memory 304, as well as any computer-readable storage medium described herein, is not to be construed as a transient signal. Memory 304, as well as any computer-readable storage medium described herein, is not to be construed as a propagating signal. Memory 304, as well as any computer-readable storage medium described herein, is to be construed as an article of manufacture.

Memory 304 may store any information utilized in conjunction with telecommunications. Depending upon the exact configuration or type of processor, memory 304 may include a volatile storage 314 (such as some types of RAM), a nonvolatile storage 316 (such as ROM, flash memory), or a combination thereof. Memory 304 may include additional storage (e.g., a removable storage 318 or a non-removable storage 320) including, for example, tape, flash memory, smart cards, CD-ROM, DVD, or other optical storage, magnetic cassettes, magnetic tape, magnetic disk storage or other magnetic storage devices, USB-compatible memory, or any other medium that can be used to store information and that can be accessed by network device 300. Memory 304 may comprise executable instructions that, when executed by processor 302, cause processor 302 to effectuate operations to map signal strengths in an area of interest.

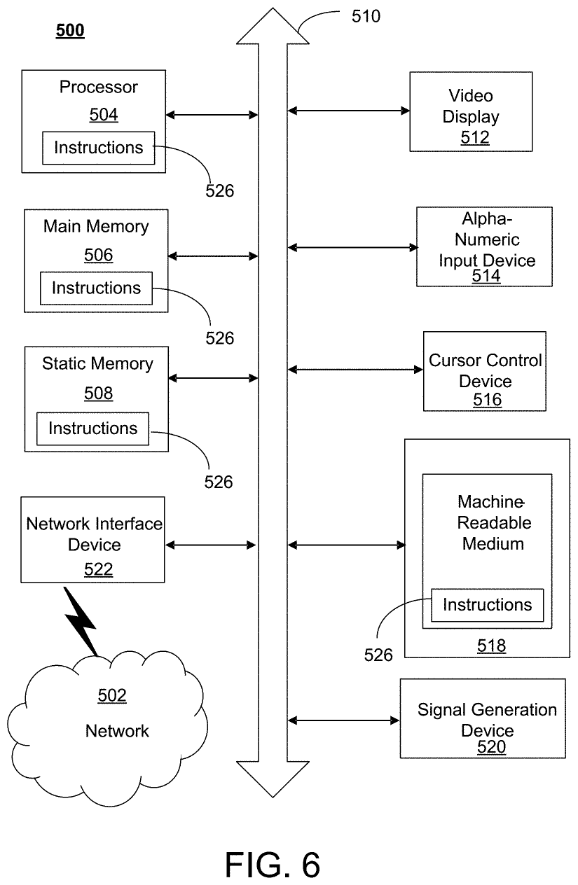

FIG. 6 depicts an exemplary diagrammatic representation of a machine in the form of a computer system 500 within which a set of instructions, when executed, may cause the machine to perform any one or more of the methods described above. One or more instances of the machine can operate, for example, as processor 302, Sensor 101, Sensor 102, base station 111, base station 113, content server 107, display 112 and other devices of FIG. 1. In some examples, the machine may be connected (e.g., using a network 502) to other machines. In a networked deployment, the machine may operate in the capacity of a server or a client user machine in a server-client user network environment, or as a peer machine in a peer-to-peer (or distributed) network environment.

The machine may comprise a server computer, a client user computer, a personal computer (PC), a tablet, a smart phone, a laptop computer, a desktop computer, a control system, a network router, switch or bridge, or any machine capable of executing a set of instructions (sequential or otherwise) that specify actions to be taken by that machine. It will be understood that a communication device of the subject disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methods discussed herein.

Computer system 500 may include a processor (or controller) 504 (e.g., a central processing unit (CPU)), a graphics processing unit (GPU, or both), a main memory 506 and a static memory 508, which communicate with each other via a bus 510. The computer system 500 may further include a display unit 512 (e.g., a liquid crystal display (LCD), a flat panel, or a solid state display). Computer system 500 may include an input device 514 (e.g., a keyboard), a cursor control device 516 (e.g., a mouse), a disk drive unit 518, a signal generation device 520 (e.g., a speaker or remote control) and a network interface device 522. In distributed environments, the examples described in the subject disclosure can be adapted to utilize multiple display units 512 controlled by two or more computer systems 500. In this configuration, presentations described by the subject disclosure may in part be shown in a first of display units 512, while the remaining portion is presented in a second of display units 512.

The disk drive unit 518 may include a tangible computer-readable storage medium on which is stored one or more sets of instructions (e.g., software 526) embodying any one or more of the methods or functions described herein, including those methods illustrated above. Instructions 526 may also reside, completely or at least partially, within main memory 506, static memory 508, or within processor 504 during execution thereof by the computer system 500. Main memory 506 and processor 504 also may constitute tangible computer-readable storage media.

As described herein, a telecommunications system may utilize a software defined network (SDN). SDN and a simple IP may be based, at least in part, on user equipment, that provide a wireless management and control framework that enables common wireless management and control, such as mobility management, radio resource management, QoS, load balancing, etc., across many wireless technologies, e.g. LTE, Wi-Fi, and future 5G access technologies; decoupling the mobility control from data planes to let them evolve and scale independently; reducing network state maintained in the network based on user equipment types to reduce network cost and allow massive scale; shortening cycle time and improving network upgradability; flexibility in creating end-to-end services based on types of user equipment and applications, thus improve customer experience; or improving user equipment power efficiency and battery life—especially for simple M2M devices—through enhanced wireless management.

While examples of a system in which unconscious behaviors and conscious behaviors for recommendations, authentication, or other actions can be processed and managed have been described in connection with various computing devices/processors, the underlying concepts may be applied to any computing device, processor, or system capable of facilitating a telecommunications system. The various techniques described herein may be implemented in connection with hardware or software or, where appropriate, with a combination of both. Thus, the methods and devices may take the form of program code (i.e., instructions) embodied in concrete, tangible, storage media having a concrete, tangible, physical structure. Examples of tangible storage media include floppy diskettes, CD-ROMs, DVDs, hard drives, or any other tangible machine-readable storage medium (computer-readable storage medium). Thus, a computer-readable storage medium is not a signal. A computer-readable storage medium is not a transient signal. Further, a computer-readable storage medium is not a propagating signal. A computer-readable storage medium as described herein is an article of manufacture. When the program code is loaded into and executed by a machine, such as a computer, the machine becomes a device for telecommunications. In the case of program code execution on programmable computers, the computing device will generally include a processor, a storage medium readable by the processor (including volatile or nonvolatile memory or storage elements), at least one input device, and at least one output device. The program(s) can be implemented in assembly or machine language, if desired. The language can be a compiled or interpreted language, and may be combined with hardware implementations.

The methods and devices associated with a telecommunications system as described herein also may be practiced via communications embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, or the like, the machine becomes a device for implementing telecommunications as described herein. When implemented on a general-purpose processor, the program code combines with the processor to provide a unique device that operates to invoke the functionality of a telecommunications system.

While the disclosed systems have been described in connection with the various examples of the various figures, it is to be understood that other similar implementations may be used or modifications and additions may be made to the described examples of a telecommunications system without deviating therefrom. For example, one skilled in the art will recognize that a telecommunications system as described in the instant application may apply to any environment, whether wired or wireless, and may be applied to any number of such devices connected via a communications network and interacting across the network. Therefore, the disclosed systems as described herein should not be limited to any single example, but rather should be construed in breadth and scope in accordance with the appended claims.

In describing preferred methods, systems, or apparatuses of the subject matter of the present disclosure—unconscious behaviors and conscious behaviors for recommendations, authentication, or other actions—as illustrated in the Figures, specific terminology is employed for the sake of clarity. The claimed subject matter, however, is not intended to be limited to the specific terminology so selected. In addition, the use of the word “or” is generally used inclusively unless otherwise provided herein.

This written description uses examples to enable any person skilled in the art to practice the claimed subject matter, including making and using any devices or systems and performing any incorporated methods. Other variations of the examples are contemplated herein.

Methods, systems, and apparatuses, among other things, as described herein may provide for the use of unconscious behaviors and conscious behaviors for recommendations, authentication, or other actions. A method, system, computer readable storage medium, or apparatus provides for sending stimulus, wherein the stimulus is in the presence of an object, wherein the stimulus comprises video, audio, or text; observing activity of the object, wherein the object comprises human or animal; measuring reaction of the object to the stimulus; classifying the reaction of the object to the stimulus; and transmitting a message based on the classification. A method, system, computer readable storage medium, or apparatus provides for detecting stimulus in the presence of a person; monitoring the person during the stimulus; recording reaction information of the person during the stimulus; classifying the reaction based on the reaction information; generating a message based on the classification; and transmitting the message. The reaction information may be sensor information that is obtained from an implanted device of the person or a wearable device of the person. All combinations in this paragraph (including the removal or addition of steps) are contemplated in a manner that is consistent with the other portions of the detailed description.

What is claimed:

1. A method comprising:
detecting stimulus in the presence of a person;
monitoring the person during the stimulus;
recording reaction information of the person during the stimulus;
classifying the reaction based on the reaction information;
generating a message based on the classification; and
transmitting the message.

2. The method of claim 1, further comprising:
determining that a threshold number of reactions have occurred or have been classified; and
based on reaching the threshold number, sending an indication of a classification trigger and corresponding action to a device near the person.

3. The method of claim 1, wherein the reaction information is obtained by an implanted device of the person or a wearable device of the person.

4. The method of claim 1, wherein the reaction information comprises unconscious behaviors.

5. The method of claim 1, wherein the reaction information comprises an unconscious behavior, wherein the unconscious behavior comprises a micro expression.

6. The method of claim 1, wherein the stimulus comprises video or text.

7. The method of claim 1, wherein the stimulus comprises audio.

8. A system comprising:
one or more processors; and
memory coupled with the processor, the memory storing executable instructions that when executed by the one or more processors cause the one or more processors to effectuate operations comprising:
detecting stimulus in the presence of a person;
monitoring the person during the stimulus;
recording reaction information of the person during the stimulus;
classifying the reaction based on the reaction information;
generating a message based on the classification; and
transmitting the message.

9. The system of claim 8, the operations further comprising:
determining that a threshold number of reactions have occurred or have been classified; and
based on reaching the threshold number, sending an indication of a classification trigger and corresponding action to a device near the person.

10. The system of claim 8, wherein the reaction information is obtained by an implanted device of the person or a wearable device of the person.

11. The system of claim 8, wherein the reaction information comprises an unconscious behavior.

12. The system of claim 8, wherein the reaction information comprises an unconscious behavior, wherein the unconscious behavior comprises a micro expression.

13. The system of claim 8, wherein the stimulus comprises video or text.

14. The system of claim 8, wherein the stimulus comprises audio.

15. A computer readable storage medium storing computer executable instructions that when executed by a computing device cause said computing device to effectuate operations comprising:
detecting stimulus in the presence of a person;
monitoring the person during the stimulus;
recording reaction information of the person during the stimulus;
classifying the reaction based on the reaction information;
generating a message based on the classification; and
transmitting the message.

16. The computer readable storage medium of claim 15, wherein the reaction information is obtained by an implanted device of the person or a wearable device of the person.

17. The computer readable storage medium of claim 15, wherein the reaction information comprises unconscious behaviors.

18. The computer readable storage medium of claim 15, wherein the reaction information comprises an unconscious behavior, wherein the unconscious behavior comprises a micro expression.

19. The computer readable storage medium of claim 15, wherein the stimulus comprises video or text.

20. The computer readable storage medium of claim 15, wherein the stimulus comprises audio.
